(19) United States
(12) Patent Application Publication
(10) Pub. No.: US 2026/0354698 A1
(43) Pub. Date: Nov. 12, 2026

(54) POLYPHASIC PHOTOPLETHYSMOGRAPHY APPARATUS

(71) Applicant: GlobalMedia Group, LLC, Scottsdale, AZ (US)

(72) Inventor: Michael D. Harris, Scottsdale, AZ (US)

(73) Assignee: GlobalMedia Group, LLC, Scottsdale, AZ (US)

(21) Appl. No.: 19/200,396

(22) Filed: May 6, 2025

(51) Int. Cl. A61B 5/024; A61B 5/026

(52) U.S. Cl. CPC A61B 5/02427; A61B 5/0261

(57) ABSTRACT

A polyphasic photoplethysmography device that allows a user to collect rapid, non-invasive health data for patients of a variety of skin colors and skin qualities using electromagnetic radiation emissions. A medical device is coupled to an apparatus employing a plurality of LED emitters to emit electromagnetic radiation of a plurality of distinct wavelengths, which is reflected off a patient's skin and detected by one or more photodetectors and at least one controller. The controller may calculate averages of the data detected to determine health parameters and predicted health parameters and to provide the health parameters and predicted health parameters to a patient or healthcare provider in real time.

### FIELD

The present disclosure generally relates to telemedicine devices, systems, and methods, and more particularly to a medical device utilizing multi-spectral photoplethysmography (PPG) to monitor health indicators and methods of using medical device.

### BACKGROUND

Photoplethysmography (“PPG”) devices enable the monitoring of important cardiovascular information in real time using optical techniques to monitor volumetric changes in blood circulation. Specifically, PPG devices allow doctors and patients to gather biometric information based on blood flow variations in a non-invasive, efficient, and inexpensive way. The use of these devices enables detection of heart rate changes that can indicate cardiac health, and even detect life-threatening cardiac events. However, the functionality and accuracy of the data derived from PPG devices is impacted by various parameters of a patient's skin (e.g., hue, saturation, brightness, tone, or any other skin parameter), due to the differing reflective properties of different skin tones. Accordingly, medical devices, systems, and methods of operation that facilitate more broadly accurate results and calculations independent of skin parameters may be desirable.

### SUMMARY

Disclosed herein is a polyphasic PPG apparatus and a medical device that is configured for home use, medical provider use, and/or use in emergency situations, such as on an airplane, on a ship, or the like. In various embodiments, the polyphasic PPG apparatus utilizes infrared light emissions to detect changes in light intensity that correspond to changes in the pulse pressure wave of an individual. In various embodiments, the PPG apparatus comprises four light sources, for example LED emitters, wherein each of the four light sources emit a distinct wavelength of light. The light sources emit the light into the skin when the apparatus is against or near an individual's skin, and it is reflected or transmitted to one or more detectors. The detector then measures the light intensity over time, and records the data. A controller may then calculate the changes in light intensity, wherein the changes in light intensity in the micro-vascular bed of tissue indicate a change in the blood flow volume.

A polyphasic PPG apparatus and medical device employing a polyphasic PPG apparatus is disclosed herein. In various embodiments, the polyphasic PPG apparatus comprises a circuit, a plurality of light sources, one or more photodetectors, and a controller.

In various embodiments, the circuit may comprise a plurality of light emitting diode (“LED”) emitters and a printed circuit board (“PCB”). In various embodiments, the plurality of LED emitters is electronically coupled to a printed circuit board. In various embodiments, each of the LED emitters may emit electromagnetic radiation in the form of light, and each LED emitter may have a nominal wavelength. In various embodiments, one or more photodetectors may also be electronically coupled to the printed circuit board. In various embodiments, the photodetector may be a sensor die.

A method is disclosed herein. In various embodiments, the method comprises attaching the polyphasic PPG apparatus to a patient, signaling the polyphasic PPG apparatus from a controller to take measurements of patient's blood flow using a plurality of LED emitters and one or more photodetectors, each of a plurality of LED emitters, each of the plurality of LED emitters configured to emit electromagnetic radiation having a nominal wavelength, the nominal wavelength for each of the plurality of LED emitters being different from a remaining of the plurality of LED emitters, receiving a signal transmitted by each of the plurality of LED emitters via the one or more photodetectors, averaging a parameter from the signal from each of the plurality of LED emitters to form an averaged parameter, calculating a predicted health parameter based on the averaged parameter, and displaying the predicted health parameter.

### BRIEF DESCRIPTION OF THE DRAWINGS

The subject matter of the present disclosure is particularly pointed out and distinctly claimed in the concluding portion of the specification. A more complete understanding of the present disclosure, however, may best be obtained by referring to the following detailed description and claims in connection with the following drawings. While the drawings illustrate various embodiments employing the principles described herein, the drawings do not limit the scope of the claims.

FIG. 1(A) illustrates a side view of a polyphasic PPG apparatus, in accordance with various embodiments.

FIG. 1(B) illustrates a side view of a polyphasic PPG apparatus in use, in accordance with various embodiments.

FIG. 2 illustrates a front view of a polyphasic PPG apparatus, in accordance with various embodiments.

FIG. 3 illustrates an exploded perspective view of a polyphasic PPG apparatus, comprising a circuit board and four LED light sources, in accordance with various embodiments.

FIG. 4(A) illustrates a side view of a polyphasic PPG apparatus with four LED lights, in accordance with various embodiments.

FIG. 4(B) illustrates a block diagram of the PPG apparatus, in accordance with various embodiments.

FIG. 5 illustrates a diagram of the reflection of light emissions in a patient's skin to a sensor, in accordance with various embodiments.

FIG. 6 illustrates a circuit comprising a barrier between a patient's skin and the circuit, in accordance with various embodiments.

FIG. 7 illustrates the movement of light emissions from four LED lights directed at a patient's skin and measured using a photodetector, in accordance with various embodiments.

FIG. 8 illustrates a flow chart of the process that corresponds to the functionality of the controller, in accordance with various embodiments.

FIG. 9 illustrates a method of use of a PPG apparatus, in accordance with various embodiments.

### DETAILED DESCRIPTION

The detailed description of various embodiments herein makes reference to the accompanying drawings and pictures, which show various embodiments by way of illustration. While these various embodiments are described in sufficient detail to enable those skilled in the art to practice the disclosure, it should be understood that other embodiments may be realized and that logical and mechanical changes may be made without departing from the spirit and scope of the disclosure. Thus, the detailed description herein is presented for purposes of illustration only and not for purposes of limitation. For example, the steps recited in any of the method or process descriptions may be executed in any order and are not limited to the order presented. Moreover, any of the functions or steps may be outsourced to or performed by one or more third parties. Modifications, additions, or omissions may be made to the systems, apparatuses, and methods described herein without departing from the scope of the disclosure. For example, the components of the systems and apparatuses may be integrated or separated. An individual component may be comprised of two or more smaller components that may provide a similar functionality as the individual component. Moreover, the operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set. Furthermore, any reference to singular includes plural embodiments, and any reference to more than one component may include a singular embodiment. Although specific advantages have been enumerated herein, various embodiments may include some, none, or all of the enumerated advantages.

Systems, methods, and computer program products are provided. In the detailed description herein, references to “various embodiments,” “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

A medical device and methods of operation of the medical device are disclosed herein. The medical device comprises a polyphasic PPG apparatus to gather data and calculate health parameters for patients with a wide range of skin colors and/or tones. Different wavelengths of light may interact with a patient's skin differently based on the various color parameters of a patient's skin, such as color (e.g., hue, saturation, brightness), a patient's skin texture, or any other unique characteristics of a patient's skin. Therefore, the polyphasic PPG apparatus utilizes at least three light emitting sources, wherein each of the at least three light emitting sources emits light of different and distinct wavelengths. The variety of wavelengths enables the polyphasic PPG apparatus, specifically a sensor or photodetector, to measure the response of the light emissions accurately on most patients, and the polyphasic PPG apparatus further comprises a controller that enables calculation of averages (or weighted averages) of the data and health parameters, enabling more health metrics and health predictors to be derived from the data collected. In various embodiments, the photodetector may be a photodiode, spectral sensor, sensor die, or other photodetector. The light captured by the photodetector may be measured using transmittances, wherein the light is transmitted through the skin, or by reflectance, wherein the light is transmitted and scattered to reflect back toward the light sources.

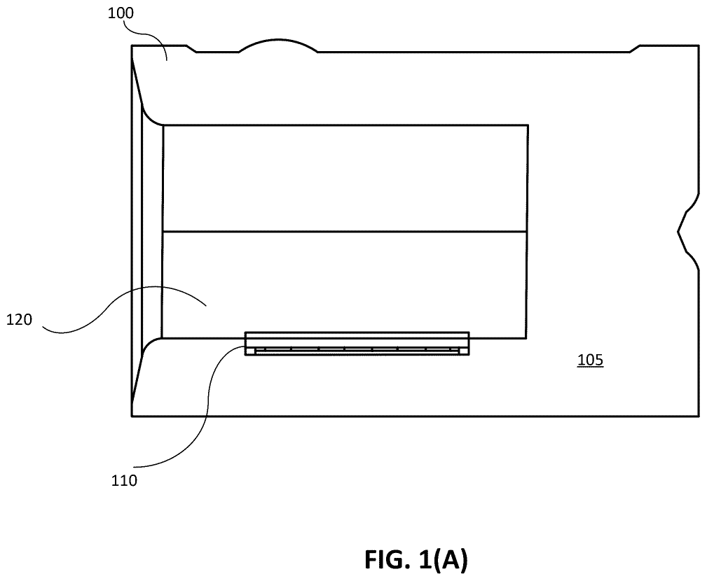

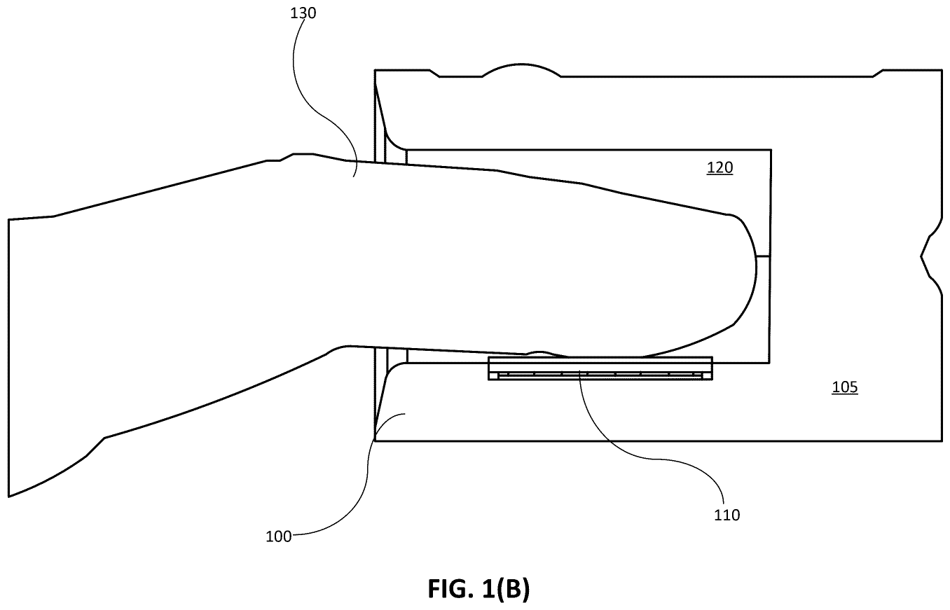

Referring now to FIG. 1(A), a cross-sectional view of a medical device 100 is shown, in accordance with various embodiments. The medical device 100 comprises a main body 105 and a polyphasic PPG apparatus 110. The main body 105 includes an aperture 120 (e.g., a blind aperture) disposed therein. Although illustrated as a unitary main body with a aperture 120 disposed therein, the present disclosure is not limited in this regard. For example, the medical device 100 can comprise a main body 105 that is separated into a top portion and a bottom portion and clamps to a user's finger and would still be within the scope of this disclosure. The aperture 120 allows a user's finger to enter the medical device 100.

The polyphasic PPG apparatus 110 is configured to be fixed to the main body 105 within the aperture 120. In this regard, a user (e.g., a patient) may contact the polyphasic PPG apparatus 110 through the aperture 120 (e.g., as shown in FIG. 1(B)). Referring now to FIG. 1(B), with like numerals depicting like elements, a user may insert a finger 130 into the aperture 120 to activate the polyphasic PPG apparatus 110 of the medical device 100. In various embodiments, the aperture 120 is configured to fit the finger of an adult, a child, or a combination of adults and children. In various embodiments, the aperture 120 can be of a fixed size or an adjustable size. For example, the aperture 120 can be adaptable to different finger sizes (e.g., via a hinged apparatus) or any other structure for coupling a device to a finger known in the medical arts and would still be within the scope of this disclosure. When a user places a finger 130 in the aperture 120, the aperture 120 may be configured to guide the finger 130 to the correct position to allow the polyphasic PPG apparatus 110 to function properly. In various embodiments, when the main body 105 includes a clamping mechanism, the medical device 100 can be configured to ensure the finger 130 is in an appropriate position upon attaching the main body 105 to the finger 130 of the user.

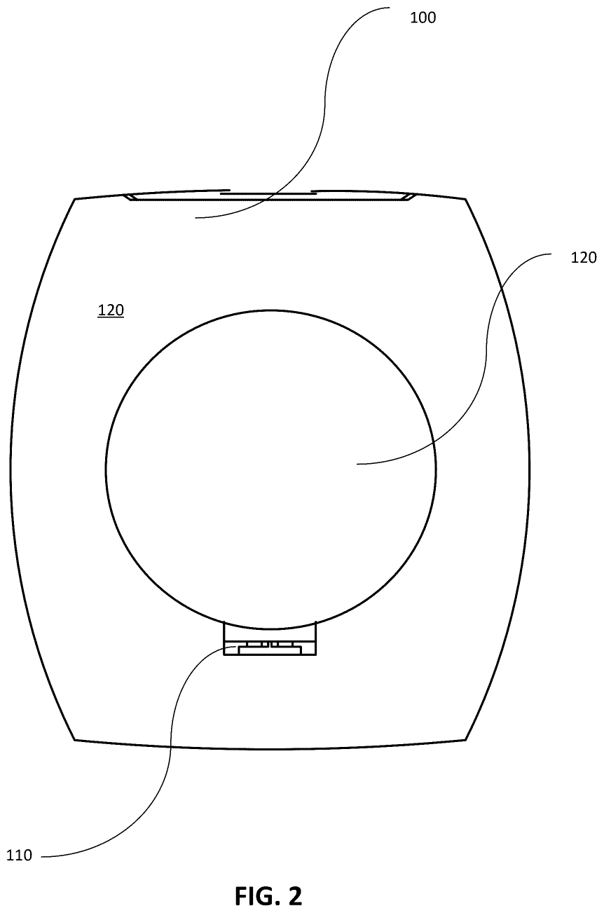

Referring now to FIG. 2, the front view of a medical device 100 is shown, wherein the aperture 120 is circular (or ovular), and the polyphasic PPG apparatus 110 is located at the bottom of the aperture 120. In various embodiments the medical device 100 may have a rounded cross-sectional shape, as in FIG. 2. In various other embodiments, the medical device 100 may have a square or circular cross-sectional shape or any other shape known in the medical arts.

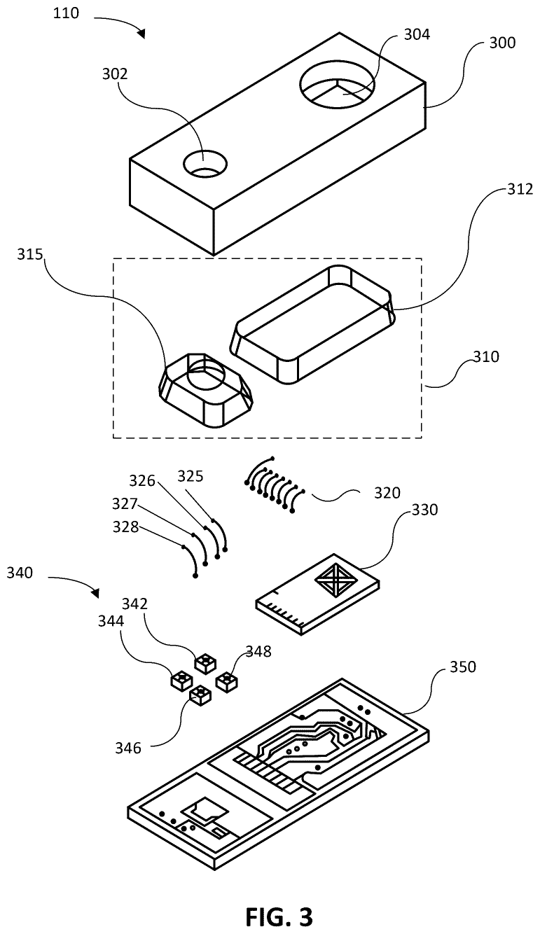

Referring now to FIG. 3, an exploded view of the polyphasic PPG apparatus 110 is depicted, comprising a printed circuit board 350, a plurality of LED emitters 340, and a sensor die 330. In various embodiments, the sensor die 330 is coupled to the printed circuit board using one or more sensor die bond wires 320. In various embodiments, the plurality of LED emitters 340 comprises a first LED emitter 342, a second LED emitter 344, a third LED emitter 346, and a fourth LED emitter 348. Although illustrated with four LED emitters, the present disclosure is not limited in this regard. For example the plurality of LED emitters 340 could comprise three LED emitters, or five or more LED emitters and still be within the scope of this disclosure. However, the polyphasic PPG apparatus 110 includes at least three LED emitters in order to facilitate a more accurate and more consistent health parameter measurements independent of various skin characteristics (e.g., color parameters, tone parameters, texture parameters, or any other skin parameter known in the art), in accordance with various embodiments.

Each of the plurality of LED emitters 340 (e.g., the first LED emitter 342, the second LED emitter 344, the third LED emitter 346, and the fourth LED emitter 348) are respectively electrically coupled to the printed circuit board 350. For example, the first LED emitter 342 can be electrically coupled to the printed circuit board 350 by a first LED bond wire 325, the second LED emitter 344 can be coupled to the printed circuit board 350 by a second LED bond wire 326, the third LED emitter 346 can be coupled to the printed circuit board 350 by a third LED bond wire 327, and the fourth LED emitter 348 can be coupled to the printed circuit board 350 by a fourth LED bond wire 328. Although described herein as being electrically coupled to the printed circuit board 350 by bond wires 325, 326, 327, 328, the present disclosure is not limited in this regard. For example, any other electrical connection known in the electrical arts for operably coupling an LED emitter to a printed circuit board 350 (or other electronic control device) is within the scope of this disclosure.

In various embodiments, the plurality of LED emitters 340 may each emit an AC component and a DC component. When the AC component is measured by the sensor die 330, the AC component may represent a change in blood volume during a patient's cardiac cycle. The DC component may allow the polyphasic PPG apparatus 110 to determine an optically detected signal from tissue of the patient. In various embodiments, each of the plurality of LED emitters 340 may each emit electromagnetic radiation with a different nominal wavelength than each of the other of the plurality of LED emitters 340. Each of the plurality of LED emitters 340 may have a wavelength between 400 nanometers (nm) and 800 nm.

In various embodiments, the nominal wavelength of a first LED emitter 342 may be between 620 nm and 750 nm. In various embodiments, the nominal wavelength of a second LED emitter 344 may be between 495 nm and 570 nm. In various embodiments, the nominal wavelength of a third LED emitter 346 may be between 450 and 495 nm. In various embodiments, the nominal wavelength of a fourth LED emitter 348 may be between 570 nm and 590 nm. The plurality of LED emitters 340 may emit colored lights, wherein the first LED emitter 342 may be red, the second LED emitter 344 may be green, the third LED emitter 346 may be blue, and the fourth LED emitter 348 may be yellow. The wavelengths of each of the emitted lights determine the color of the emitted light of each of the plurality of LED emitters 340. The wavelengths of each of the emitted lights also determine the depth to which the emitted light penetrates beneath the surface of a patient's skin before reflecting.

As shown in FIG. 3, the polyphasic PPG apparatus 110 may comprise a cap 300, which may house a transparent mold 310. Although described herein as being transparent, the transparent mold 310 is not limited in this regard. For example, the transparent mold 310 could be a translucent mold between 50% and 100% transparency, or between 70% and 100% transparency, or between 90% and 100% transparency and would still be within the scope of this disclosure. Similarly, although described herein as a “mold,” the transparent disclosure is not limited in this regard. For example, the transparent mold 310 could be made from other manufacturing methods besides molding and would still be within the scope of this disclosure, such as machining or additive manufacturing. The transparent mold 310 may be a single piece, or may be multiple pieces as illustrated in FIG. 3.

In various embodiments, the transparent mold 310 may comprise a LED transparent mold 315 that covers the plurality of LED emitters 340 and a sensor die transparent mold 312 that covers the sensor die 330. The LED transparent mold 315 may be configured with an opening to allow the plurality of LED emitters 340 to emit light outside of the LED transparent mold 315. In various embodiments, the LED transparent mold 315 and the sensor die transparent mold 312 may be made of a polymeric material. In various embodiments, the LED transparent mold 315 and a sensor die transparent mold 312 may have a thickness between 0.5 mm and 1.5 mm. The polyphasic PPG apparatus 110 may further comprise a cap 300. The cap 300 may comprise a first cap opening 302 and a second cap opening 304. In various embodiments, the cap 300 may be liquid crystal polymer (LCP) cap. However, the present disclosure is not limited in this regard and various potential materials for the cap 300 may be readily apparent to one skilled in the art and would be within the scope of this disclosure.

The cap 300 may cover and/or contain the plurality of LED emitters 340 and the sensor die 330. In various embodiments, the cap 300 may cover only the plurality of LED emitters 340. In various embodiments, the cap 300 may cover only the sensor die 330. In various embodiments, LED transparent mold 315 and the sensor die transparent mold 312 may fit into the cap 300. In various embodiments, the cap 300 may be coupled to a transparent mold 310, wherein the transparent mold 310 may be affixed to the inside of the cap 300. The transparent mold 310 may be configured to be in contact with a patient's skin when the polyphasic PPG apparatus 110 is in use. In various embodiments, the cap 300 may be configured to prevent escaping light from the plurality of LED emitters 340 from affecting the data collected by the sensor die 330. In various embodiments, the plurality of LED emitters 340 and the sensor die 330 may be configured to be located between the printed circuit board 350 and the cap 300. In various embodiments, the transparent mold 310 may further contain at least one magnifying lens, wherein the magnifying lenses are fixed to the transparent mold in a configuration allowing each of the plurality of LED emitters 340 to align with one of the magnifying lenses.

The first cap opening 302 may align with the plurality of LED emitters 340 and the second cap opening 304 may align with the sensor die 330. The cap 300 may prevent light from scattering when electromagnetic radiation is emitted from the plurality of LED emitters 340 and when it is entering the sensor die 330. Additionally, the cap 300 may prevent ambient light interfering with the sensor die 330 readings and focus the electromagnetic radiation emitted by the plurality of LED emitters 340 into the patient's skin. Preventing light from scattering by focusing the electromagnetic radiation may improve the quality of data obtained from the polyphasic PPG apparatus 110.

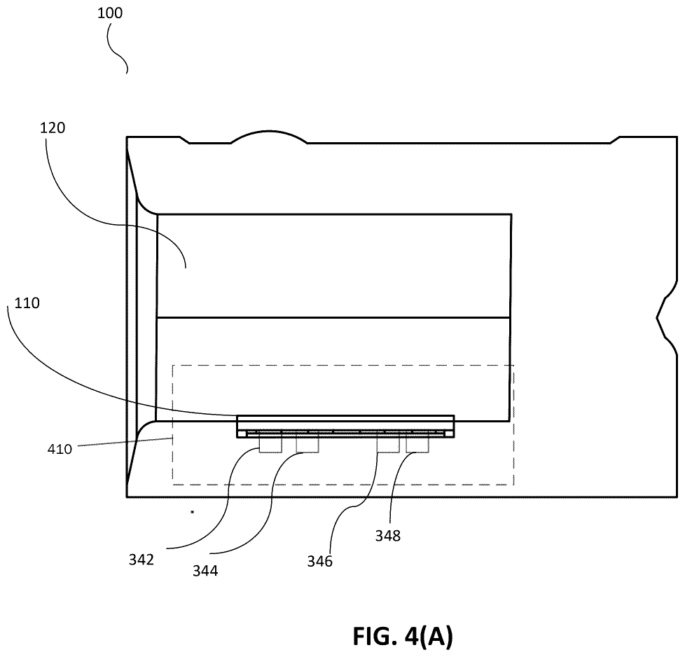

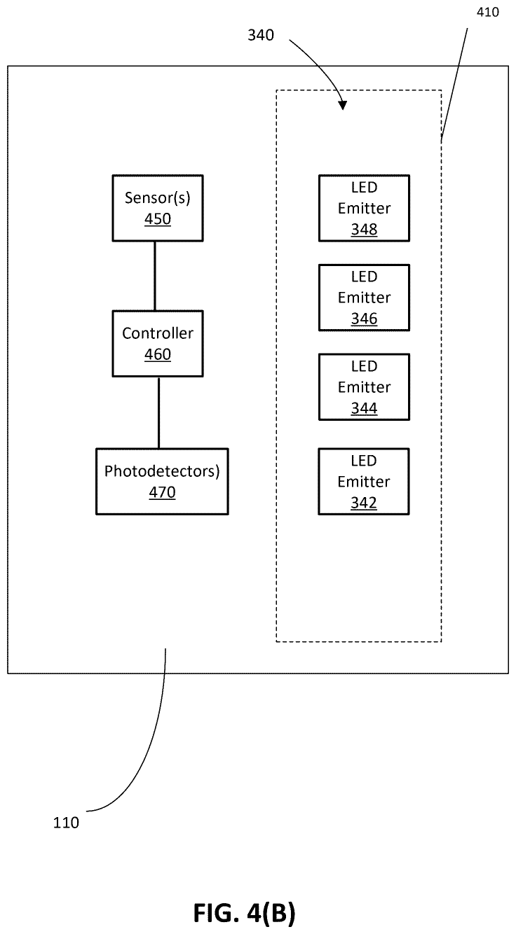

Referring now to FIG. 4(A), a diagram of the medical device 100, comprising the polyphasic PPG apparatus 110 is shown. The polyphasic PPG apparatus 110 may be coupled to the medical device 100 on the bottom surface of an aperture 120, wherein the aperture 120 is configured to fit a human finger. In various embodiments, the polyphasic PPG apparatus 110 may comprise at least three LED emitters. In various embodiments, the polyphasic PPG apparatus 110 comprises four LED emitters (e.g., the first LED emitter 342, the second LED emitter 344, the third LED emitter 346, and the fourth LED emitter 348). In various embodiments, the polyphasic PPG apparatus 110 comprises an LED array 410 may comprise four LED emitters arranged linearly. In various embodiments, the LED array 410 may comprise more than four LED emitters. FIG. 4(B) shows LED array 410, wherein the first LED emitter 342, the second LED emitter 344, the third LED emitter 346, and the fourth LED emitter 348 are arranged in a line. However, the present disclosure is not limited in this regard and various arrangements of the plurality of LED emitters 340 is within the scope of this disclosure. For example, in various embodiments, the LED array 340 could be arranged in pairs of two (e.g., two rows and two columns).

Referring to FIG. 4(B), the polyphasic PPG apparatus 110 comprises one or more sensors 450, a controller 460, and one or more photodetectors 470. In various embodiments, the one or more sensors 450, the controller 460, and the one or more photodetectors 470 may be electronically connected, wherein data collected by the one or more sensors 450 and the one or more photodetectors 470 may be automatically transmitted to the controller 460. In various embodiments, the data collected by each of the one or more sensors 450 and each of the one or more photodetectors 470 may be wirelessly transmitted to the controller 460. In various embodiments, the data collected by each of the one or more sensors 450 and the one or more photodetectors 470 may be transmitted to the controller 460 using a wired connection (e.g., through the printed circuit board 350 from FIG. 3). The present disclosure is not limited in this regard.

In various embodiments, the controller 460 can be integrated into a computer system of the medical device 100 from FIG. 1(A) and 1(B) (e.g., a printed circuit board 350 as shown in FIG. 3). In various embodiments, the controller 460 can be configured as a central network element or hub to access various systems and components of printed circuit board 350 from FIG. 3. Controller 460 can comprise a network, computer-based system, and/or software components configured to provide an access point to various systems and components of a control system for the medical device 100 from FIG. 1(A) and 1(B). In various embodiments, controller 460 comprises one or more processors. In various embodiments, controller 460 can be implemented in a single processor of the one or more processors. In various embodiments, controller 460 can be implemented as and may include more than one of the one or more processors and/or one or more tangible, non-transitory memories and be capable of implementing logic. Each of the one or more processors can be a general purpose processor, a digital signal processor (“DSP”), an application specific integrated circuit (“ASIC”), a field programable gate array (“FPGA”) or other programable logic device, discrete gate or transistor logic, discrete hardware components, or the one or more processors can be any combination thereof. Controller 460 can comprise a processor in the one or more processors that is configured to implement various logical operations in response to execution of instructions, for example, instructions stored on a non-transitory, tangible, computer-readable medium configured to communicate with the processor in the one or more processors.

In various embodiments, the one or more sensors 450 can comprise any sensor known in the medical arts to facilitate a start of a measurement process. For example, the one or more sensors 450 can comprise a temperature sensor (e.g., where measurements are used by the controller 460 to determine a finger is in contact with the polyphasic PPG apparatus 110 from FIG. 1(A) and 1(B)), a pressure sensor (e.g., where measurements are used by the controller 460 to determine a finger is in contact with the polyphasic PPG apparatus 110 from FIG. 1(A) and 1(B)), or any other sensor that could be utilized to determine that a finger is disposed in the aperture 120 of the medical device 100 from FIG. 1(A) and 1(B), in accordance with various embodiments. In various embodiments, the controller 460 is coupled to the printed circuit board 350 from FIG. 3. In this regard, the controller 460 is configured to receive data from the plurality of LED emitters 340 through the one or more photodetectors 470. The controller 460 may be configured to take the data received from the one or more photodetectors 470 and perform various calculations to determine health parameters as described further herein. In various embodiments, the controller 460 may be configured to calculate averages or weighted averages of the health parameters to determine an accurate snapshot of the health parameters over time. In various embodiments, the one or more photodetectors 470 can comprise a single photodetector, a detector corresponding to each emitter (e.g., four photodetectors for a four LED emitter embodiment). However, the present disclosure is not limited in this regard.

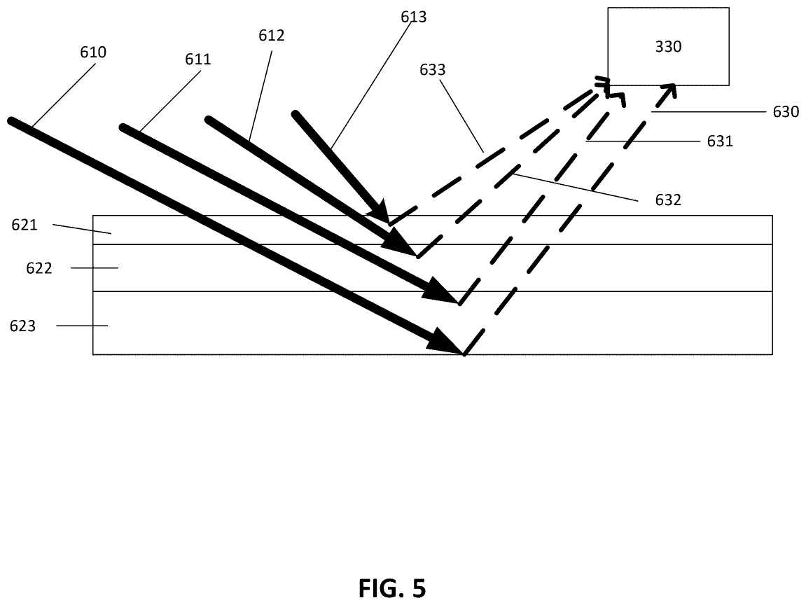

Referring to FIG. 5, the behavior of the light transmissions of each for four LED emitters when directed at a patient's skin is shown. Generally, light emissions with longer wavelengths penetrate deeper into a patient's skin. The patient's skin comprises several layers, including the layer closest to the surface, the stratum corneum epidermis 621, the papillary dermis 622 below that, and the deepest layer illustrated is the reticular dermis 623. The stratum corneum epidermis 621 contains melanin, and the papillary dermis 622 contains hemoglobin. In various embodiments, as described previously herein, the first LED emitter may emit a wavelength of between 620 and 750 nm (e.g., first light emission 610), a second LED emitter may emit a wavelength between 495 nm and 570 nm (e.g., second light emission 611), a third LED emitter may emit a wavelength between 450 and 495 nm (e.g., third light emission 612), and a fourth LED emitter may emit a wavelength between 570 nm and 580 nm (e.g., fourth light emission 613).

In various embodiments, when the polyphasic PPG apparatus is activated against a patient's skin, the first LED emitter may emit the first light emission 610, which may travel to the reticular dermis 623 before exiting the patient's skin as a first exiting emission 630 and being captured by a sensor die 330 (e.g., one of the one or more photodetectors 470 from FIG. 4(B)). A second LED emitter may emit a second light emission 611, which may travel to the papillary dermis 622 before exiting the patient's skin as a second exiting emission 631 and being captured by the sensor die 330 (e.g., one of the one or more photodetectors 470 from FIG. 4(B)). A third LED emitter may emit a third light emission 612, which may travel to the stratum corneum epidermis 621 before exiting the patient's skin as a third exiting emission 632 and being captured by the sensor die 330 (e.g., one of the one or more photodetectors 470 from FIG. 4(B)). A fourth LED emitter may emit a fourth light emission 613, which may travel to just below the surface of the patient's before exiting the patient's skin as a fourth exiting emission 633 and being captured by the sensor die 330. In various embodiments, there may be greater than four LED emitters. In various embodiments, the various parameters of a patient's skin may change the depth each of the light emissions reached below the surface of the patient's skin.

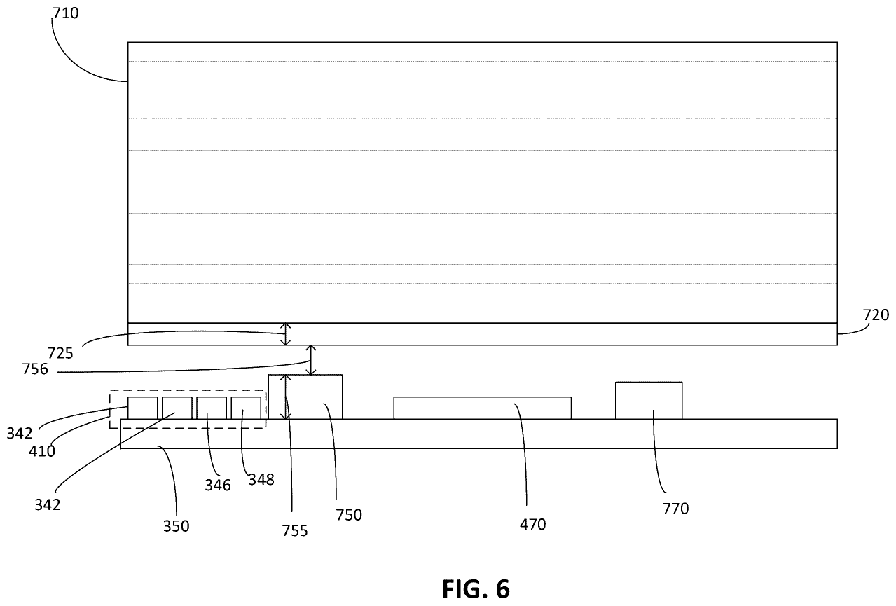

Referring now to FIG. 6, the polyphasic PPG apparatus 110 may comprise an optical barrier 720 that covers the LED light array, one or more photodetectors 470, and the printed circuit board 350. The optical barrier 720 may be a glass cover with a thickness of between 0.5 millimeters and 1.5 millimeters. However, the present disclosure is not limited in this regard and various thicknesses for the optical barrier 720 would be within the scope of this disclosure.

In various embodiments, the optical barrier 720 may be separated from the printed circuit board 350 using a first barrier 750 and a second barrier 770. The first barrier 750 and second barrier 770 are configured to ensure the optical barrier 720 remains an appropriate distance from the LED array 410 and the one or more photodetectors 470. Additionally, the first barrier 750 and second barrier 770 may be configured to prevent the one or more photodetectors 470 from receiving any stray light directly from the LED array 410. The first barrier 750 and the second barrier 770 may have a height 755. The height 755 may be approximately double the thickness 725 of the optical barrier 720 and must be larger than the thickness 725 of the optical barrier 720. The optical barrier 720 may also be separated from the printed circuit board 350 by an air gap 756, wherein the optical barrier 720 is suspended above the LED array 410 and the one or more photodetectors 470 such that there is an air gap 756. The optical barrier 720 may be a transparent mold 310. The optical barrier 720 may be in contact with the user's skin 710 when the polyphasic PPG apparatus 110 is in use.

In various embodiments, LED array 410 may be coupled to the printed circuit board 350 at the top of the printed circuit board 350, and the one or more photodetectors 470 may be coupled to the printed circuit board 350 in the center of the printed circuit board 350, as shown in FIG. 6. In various other embodiments, the LED array 410 may be coupled to the printed circuit board 350 in the center of the printed circuit board 350, and the one or more photodetectors 470 may be coupled to the printed circuit board 350 at the top or bottom end of the printed circuit board 350. The present disclosure is not limited in this regard, and various different arrangements may be readily apparent to one skilled in the art.

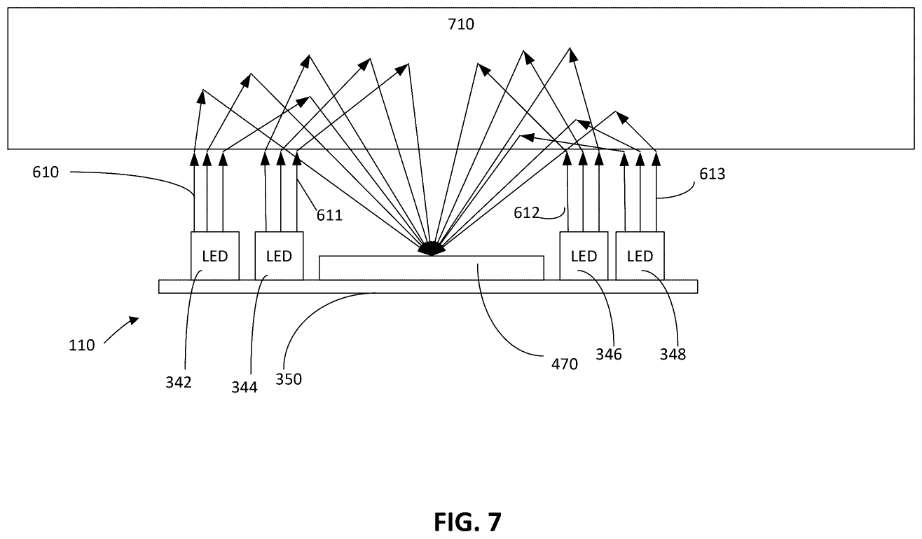

Referring now to FIG. 7, which illustrates a visual representation of LED light emissions traveling to a patient's skin 710 and to one or more photodetectors 470, in accordance with various embodiments of the polyphasic PPG apparatus 110. In the configuration shown in FIG. 7, the four LED emitters are configured wherein the first LED emitter 342 and the second LED emitter 344 are on a first side of the one or more photodetectors 470, and the third LED emitter 346 and the fourth LED emitter 348 are on a second side of the one or more photodetectors 470. Each of the LED emitters may emit light at a different frequency when the polyphasic PPG apparatus 110 is activated and in position against a patient's skin 710. A first LED emitter 342 emits a first light emission 610 with a first frequency, a second LED emitter 344 emits a second light emission 611, a third LED emitter 346 emits a third light emission 612, and a fourth LED emitter 348 emits a fourth light emission 613. The first light emission 610 reaches the surface of the patient's skin 710 and is deflected at an angle before traveling to a depth in the patient's skin 710. The first light emission 610 then travels out of the skin 710 and reaches the one or more photodetectors 470. The second light emission 611, third light emission 612, and fourth light emission 613 all similarly penetrate the skin 710 and travel at distinct angles to distinct depths beneath the surface of the patient's skin 710, wherein each distinct depth depends on the wavelength of each light emission, before exiting the patient's skin 710 and reaching the one or more photodetectors 470. Each of the first light emission 610, second light emission 611, third light emission 612, and fourth light emission 613 have different wavelengths and travel to different depths beneath the surface of the patient's skin 710 as described previously herein.

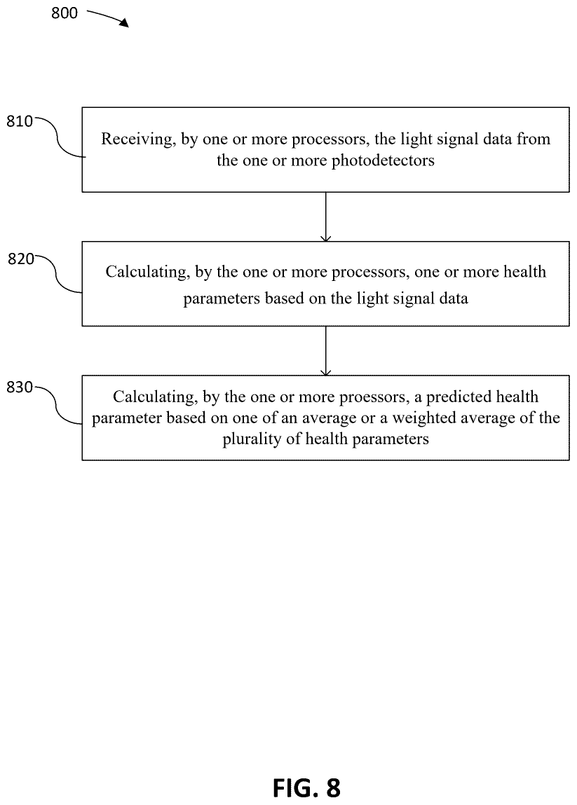

Referring now to FIG. 8, a flow chart of the process 800 that corresponds to the functionality of the controller 460 from FIG. 6 is shown, in accordance with various embodiments. The process 800 comprises receiving, by one or more processors, light signal data from each of the plurality of LED emitters through one or more photodetectors 470 (step 810). The process 800 further comprises calculating, by the one or more processors (e.g., the controller 460), one or more health parameters based on the light signal data (step 820). The process 800 further comprises calculating a predicted health parameter based on one of an average or a weighted average of the plurality of health parameters (step 830).

The controller obtains data from the one or more photodetectors, which have collected data from the light emissions from the plurality of LED emitters. The controller then receives the light signal data from the one or more photodetectors (process 800). Referring back to FIG. 4(B), the controller 460, may be electronically coupled to the LED array 410 and configured to receive raw data from the one or more sensors 450 and/or the one or more photodetectors 470. In various embodiments, the process 800 can be initiated in response to the controller 460 determining a finger 130 is disposed within the aperture 120 of the medical device 100 from FIG. 1(A) and 1(B) as described previously herein.

In various embodiments, there may be more than one of the one or more sensors 450 and more than one of the one or more photodetectors 470. In various embodiments, the controller 460 receives raw light signal data from each of the one or more photodetectors 470. The raw light signal data may comprise an AC component and a DC component and light intensity measurements from the transmitted light (illustrated as exiting emissions 630, 631, 632, and 633 in FIG. 5) reflected off of different layers within the patient's skin.

Returning to FIG. 8, the controller then may calculate at least one health parameter based on the light signal data from the one or more photodetectors (step 810). The controller may be configured to account for the air gap 756 and optical barrier 720, shown in FIG. 6, in calculating the at least one health parameter. The health parameters may include measurements determining a patient's circulatory health which may enable early detection of anomalies and allow patients to make informed medical decisions. In various embodiments, health parameters may include heart rate, blood pressure, pulse oxygen levels, and other health indicators that may be determined by blood flow volume changes.

In various embodiments, the controller may calculate at least one predicted health parameter based on the average or weighted average of health parameters calculated (step 820). The weighted averages of the health parameters allow patients and users of the polyphasic PPG apparatus to observe trends and patterns in health parameters and potentially predict health changes and problems.

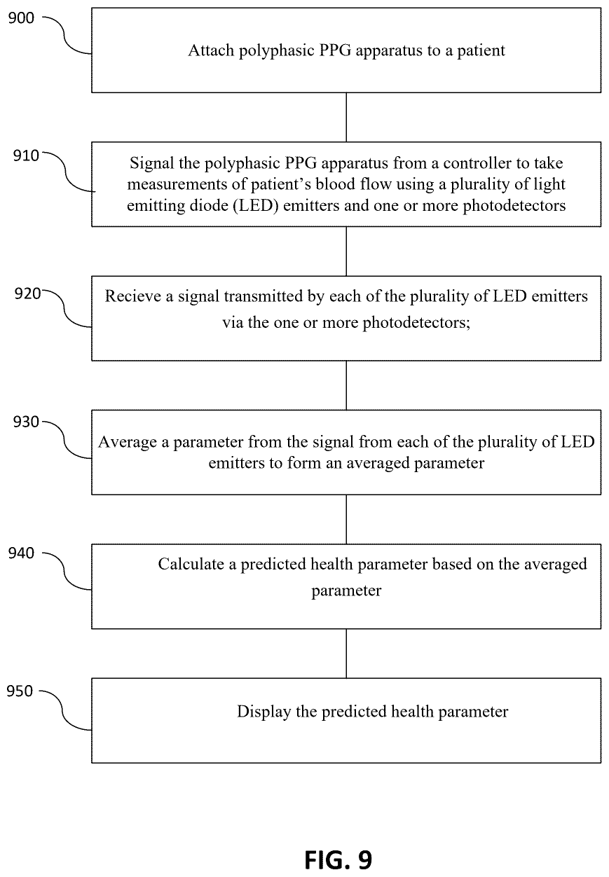

Referring now to FIG. 9, a method 900 for using the polyphasic PPG apparatus is shown. First, a healthcare provider may attach the polyphasic PPG apparatus to the patient. In various embodiments, a patient may attach the polyphasic PPG apparatus to his or herself. As shown in FIG. 1(A) and 1(B), attaching the polyphasic PPG apparatus to a patient may comprise inserting a patient's finger 130 into an aperture 120 of medical device 100. In various embodiments, the medical device 100 may comprise a hinge to clamp over the patient's finger 130. In various other embodiments, the aperture 120 may be stationary and the patient's finger 130 may be moved inside the aperture 120 and rest on the bottom of the aperture 120 where the polyphasic PPG apparatus 110 is located.

Referring again to FIG. 9, a method 900 of use of the polyphasic PPG apparatus comprises signaling the polyphasic PPG apparatus from a controller to take measurements of patient's blood flow by signaling the polyphasic PPG apparatus from a controller to take measurements of patient's blood flow using a plurality of light emitting diode (LED) emitters and one or more photodetectors (step 910). In various embodiments, the controller may be activated to turn on the LED emitters and activate the one or more photodetectors. The LED transmitters may transmit electromagnetic radiation (light) of different frequencies toward the patient's finger, and the one or more photodetectors may detect the electromagnetic radiation reflected from the patient's finger.

In various embodiments, the signal received by each of the plurality of LED emitters and captured by the one or more photodetectors may then be transmitted to the controller (step 920). The controller may be electronically coupled to the one or more photodetectors. In various embodiments, the controller is electronically coupled to the one or more photodetectors using a wireless connection. In other various embodiments, the controller is electronically coupled to the photodetectors using a wired connection. In various embodiments, the LED emitters may also be electronically coupled to the controller. The direct connection between the LED emitters and the controller may enable data related to the original intensity and wavelengths of the electromagnetic radiation emitted from the LED emitters to be provided directly to the controller.

The controller may then calculate a parameter from the signal from each of the plurality of LED emitters (step 930). The controller may then calculate an averaged parameter to determine a predicted health parameter based on the averaged parameter, in accordance with various embodiments (step 940). In various embodiments, the controller may calculate the average of the electromagnetic radiation data from each of the plurality of LED emitters. In various embodiments, the controller may be programmed to remove outliers from the data to improve the accuracy and quality of the health parameters. In various embodiments, the controller may be programmed to weight the averages of each LED emitter differently based on the color of the patient's skin and the wavelength of each of the emissions from each LED emitter. For example, where electromagnetic radiation with a higher wavelength penetrates darker skin more effectively, the LED emitter that emits the electromagnetic radiation with the highest wavelength may be given more weight by the controller when it is calculating health parameters.

In various embodiments, the polyphasic PPG apparatus measures an AC and a DC component, wherein the DC component corresponds to an optically detected signal from a tissue of the patient. In various embodiments, health parameters may be calculated by the controller by calculating a component ratio of the data using both the AC component of the signal and the DC component of the signal captured by the photodetector. In various embodiments, the component ratio may be found using the signal for each of the plurality of LED emitters. In various embodiments, an averaged health parameter may be an average of the component ratio for each of the plurality of LED emitters. In various embodiments, averaged health parameters may be used to determine a predicted health parameter. In various embodiments, a predicted health parameter is a volumetric variation of blood circulation of the patient.

In various embodiments, the polyphasic PPG apparatus may display the predicted health parameter (step 950). The display may be a graphic screen or LED display embedded in a medical device, or the polyphasic PPG apparatus may connect to a smartphone or smartwatch using a Bluetooth or wired connection. In various embodiments, the polyphasic PPG apparatus may display various calculated data, for example, graphs showing health information average and health information over time, alerts when health information indicates a significant change or a dangerous medical situation, and other numerical data.

The process flows depicted are merely embodiments and are not intended to limit the scope of the disclosure. For example, the steps recited in any of the method or process descriptions may be executed in any order and are not limited to the order presented. It will be appreciated that the following description makes appropriate references not only to the steps and user interface (UI) elements, but also to the various system components as described herein. It should be understood that, although exemplary embodiments are illustrated in the figures and described herein, the principles of the present disclosure may be implemented using any number of techniques, whether currently known or not. The present disclosure should in no way be limited to the exemplary implementations and techniques illustrated in the drawings and described below. Unless otherwise specifically noted, articles depicted in the drawings are not necessarily drawn to scale.

Benefits, other advantages, and solutions to problems have been described herein regarding specific embodiments. Furthermore, the connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B, and C may be present in a single embodiment; for example, A and B, A and C, B and C, or A and B and C. Different cross-hatching is used throughout the figures to denote different parts but not necessarily to denote the same or different materials.

Systems, methods, and apparatus' are provided herein. In the detailed description herein, references to “one embodiment,” “an embodiment,” “various embodiments,” etc., indicate that the embodiment described may include a particular feature, structure, or characteristic, but every embodiment may not necessarily include the feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element herein is to be construed under the provisions of 35 U.S.C. 212(f) unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

Finally, any of the above-described concepts can be used alone or in combination with any or all the other above-described concepts. Although various embodiments have been disclosed and described, one of ordinary skill in this art would recognize that certain modifications would come within the scope of this disclosure. Accordingly, the description is not intended to be exhaustive or to limit the principles described or illustrated herein to any precise form. Many modifications and variations are possible considering the above teaching.

What is claimed is:

1. A polyphasic photoplethysmography (“PPG”) apparatus, comprising:
a circuit comprising a plurality of light emitting diode (LED) emitters connected to a printed circuit board, the circuit configured to determine a volumetric variation of blood circulation of a patient, each of the plurality of LED emitters configured to emit electromagnetic radiation having a nominal wavelength, the nominal wavelength for each of the plurality of LED emitters being different from a remaining of the plurality of LED emitters;
one or more photodetectors, each of the one or more photodetectors electronically connected to the printed circuit board, wherein the one or more photodetectors is configured to capture light signals from each of the plurality of LED emitters; and
a controller in electronic communication with the plurality of LED emitters and the one or more photodetectors, the controller configured to:
receive light signal data from each of the plurality of LED emitters through the one or more photodetectors;
calculate a plurality of health parameters, each of the plurality of health parameters associated with one of the plurality of LED emitters; and
calculate a predicted health parameter based on one of an average or a weighted average of the plurality of health parameters.

2. The polyphasic PPG apparatus of claim 1, further comprising:
a liquid crystal polymer (LCP) cap attached to the printed circuit board, the LCP cap containing the plurality of LED emitters and the one or more photodetectors; and
a transparent mold affixed to the inside of the LCP cap where the plurality of LED emitters and the one or more photodetectors are located between the printed circuit board and the LCP cap.

3. The polyphasic PPG apparatus of claim 2, wherein the transparent mold contains a plurality of magnifying lenses.

4. The polyphasic PPG apparatus of claim 3, wherein each of the plurality of magnifying lenses is situated over each of the plurality of LED emitters.

5. The polyphasic PPG apparatus of claim 1, wherein:
the nominal wavelength of a first of the plurality of LED emitters is between 620 nm and 750 nm;
the nominal wavelength of a second of the plurality of LED emitters is between 495 nm and 570 nm;
the nominal wavelength of a third of the plurality of LED emitters is between 450 and 495 nm; and
the nominal wavelength of a fourth of the plurality of LED emitters is between 570 nm and 580 nm.

6. The polyphasic PPG apparatus of claim 2, wherein the light signal data includes at least one of transmittance data and reflectance data for each of the plurality of LED emitters.

7. The polyphasic PPG apparatus of claim 6, wherein the transparent mold has a predetermined thickness.

8. The polyphasic PPG apparatus of claim 7, wherein the transparent mold is configured to contact a patient's skin during operation of the polyphasic PPG apparatus.

9. The polyphasic PPG apparatus of claim 8, wherein the LCP cap is configured to prevent escaping light from the plurality of LED emitters from affecting the reading of the one or more photodetectors.

10. The polyphasic PPG apparatus of claim 9, wherein the transparent mold is suspended above the plurality of LED emitters and the one or more photodetectors such that there is a predetermined air-gap.

11. A method for using a polyphasic photoplethysmography (“PPG”) apparatus, the method comprising:
attaching the polyphasic PPG apparatus to a patient;
signaling the polyphasic PPG apparatus from a controller to take measurements of patient's blood flow using a plurality of light emitting diode (LED) emitters and one or more photodetectors, each of a plurality of LED emitters, each of the plurality of LED emitters configured to emit electromagnetic radiation having a nominal wavelength, the nominal wavelength for each of the plurality of LED emitters being different from a remaining of the plurality of LED emitters;
receiving a signal transmitted by each of the plurality of LED emitters via the one or more photodetectors;
averaging a parameter from the signal from each of the plurality of LED emitters to form an averaged parameter;
calculating a predicted health parameter based on the averaged parameter; and
displaying the predicted health parameter.

12. The method of claim 11, wherein:
the nominal wavelength of a first of the plurality of LED emitters is between 620 nm and 750 nm;
the nominal wavelength of a second of the plurality of LED emitters is between 495 nm and 570 nm.

13. The method of claim 12, wherein:
the nominal wavelength of a third of the plurality of LED emitters is between 450 and 495 nm; and
the nominal wavelength of a fourth of the plurality of LED emitters is between 570 nm and 580 nm.

14. The method of claim 11, wherein the signal from the polyphasic PPG apparatus contains an AC component and a DC component.

15. The method of claim 14, wherein the AC component of the signal from the polyphasic PPG apparatus represents a change in blood volume during a cardiac cycle of the patient.

16. The method of claim 14, wherein the DC component of the signal from the polyphasic PPG apparatus corresponds to an optically detected signal from a tissue of the patient.

17. The method of claim 16, wherein the parameter of the signal from the polyphasic PPG apparatus is a component ratio using both the AC component of the signal and the DC component of the signal.

18. The method of claim 17, wherein the component ratio is found using the signal for each of the plurality of LED emitters.

19. The method of claim 18, wherein the averaged parameter is an average of the component ratio for each of the plurality of LED emitters.

20. The method of claim 19, wherein the predicted health parameter is a volumetric variation of blood circulation of the patient.
